(19) United States
(12) Patent Application Publication
(10) Pub. No.: US 2006/0110374 A1
(43) Pub. Date: May 25, 2006

(54) Method to accelerate stem cell recruitment and homing

(76) Inventors: Dudy Czeiger, Lehavim (IL); Valentin Fulga, Tel Aviv (IL); Yael Porat, Hod Hasharon (IL); Daniel Goldstein, Efrat (IL)

Correspondence Address: COOPER & DUNHAM, LLP, 1185 AVENUE OF THE AMERICAS, NEW YORK, NY, 10036, US

(21) Appl. No.: 11/271,043

(22) Filed: Nov. 11, 2005

Related U.S. Application Data
(60) Provisional application No. 60/631,098, filed on Nov. 24, 2004.

(51) Int. Cl. A61K 35/12; A61N 1/00

(52) U.S. Cl. 424/93.7; 607/2

(57) ABSTRACT

A method is provided for facilitating treatment of a patient, including stressing a portion of the patient to an extent sufficient to induce homing of progenitor cells to the portion of the patient. A method is additionally provided for use with tissue of a patient, including evaluating an indication of a level of stromal cell-derived factor-1 (SDF-1) in the tissue, and determining an indication of a number of stem cells in the tissue responsive to the indication of the level of SDF-1. A method is yet additionally provided for use with tissue of a patient, including evaluating an indication of a level of SDF-1 in the tissue, and determining an indication of a level of stress of a portion of the patient, responsive to the indication of the level of SDF-1. Other embodiments are also described.

### CROSS-REFERENCES TO RELATED APPLICATIONS

This application claims the benefit of U.S. Provisional Patent Application 60/631,098, filed Nov. 24, 2004, entitled, “Method to accelerate stem cell recruitment and homing,” which is assigned to the assignee of the present patent application and is incorporated herein by reference.

### BACKGROUND OF THE INVENTION

Stem cells are defined by their ability to self renew, and to form one or more differentiated cell types. One division of the stem cell family is between those isolated from the embryo, known as embryonic stem (ES) cells, and those found in adult somatic tissue, known as adult stem cells. Adult stem cells are considered to be multipotent stem cells, meaning that they are capable of producing a limited range of differentiated cell lineages according to their location. However, it currently appears that certain adult stem cells, removed from their usual location, transdifferentiate into cells that reflect their new environment. Adult stem cells are found in many tissues and organs where they have the capacity to replenish cells that are lost during physiological homeostasis.

The property of some adult stem cells in which they appear to undergo a process of transdifferentiation, or in which they exhibit plasticity, has led to significant interest in these cells. Plasticity describes a property of adult stem cells whereby they are able to produce specialized cells that are outside of their normal lineage commitment. In vitro and/or in vivo studies have demonstrated that these cells can transdifferentiate into various tissue cells when placed under specific conditions.

Hematopoietic stem cells (HSC) are functionally defined as cells capable of reconstituting and maintaining all blood lineages. These cells are usually defined by various cell surface markers. Cells expressing certain combinations of markers can behave as HSCs. Using a relevant technique, e.g., antibody and fluorescence-activated cell sorting (FACS) or magnetic-assisted cell sorting (MACS), these cells can be isolated for research purposes or transplantation.

In vivo studies have shown transdifferentiation of HSCs to cardiomyocytes and to vascular structures. These studies have also demonstrated improvement in cardiac function following intravenous administration of HSCs.

An alternative method for HSC delivery to the damaged heart utilizes enhancement of the process of migration and homing from the bone marrow. Mobilization of HSCs using stem cell factor (SCF) and granulocyte colony stimulating factor (G-CSF) before and after myocardial infarction in a mouse model was seen to significantly increase survival and cardiac function. Sections of heart from treated mice revealed newly formed myocytes and blood vessels (1) (Citation information for numbered references appears hereinbelow.) A second study, in non-human primates, that infused SCF and G-CSF 4 hours post-MI, was able to demonstrate regeneration of vascular structures, with a significant increase in the number of capillaries and arterioles. In contrast to the former study, using the mouse model, formation of myocytes was not observed and there was no benefit to cardiac function. (2) The first human trial of stem cell mobilization to treat coronary artery disease used a protocol of intra-coronary injection of granulocyte-macrophage colony-stimulating factor, followed by 2 weeks of subcutaneous administration. Treated patients had a significant increase in coronary collateral flow, suggesting new vessel formation. (3)

Mesenchymal stem cells (MSCs) are found in bone marrow, muscle, skin and adipose tissue. MSCs are characterized by the potential to differentiate into muscle, fibroblasts, bone, tendon, ligament and adipose tissue. MSCs have been used as well in cardiac research, mainly derived from bone marrow heterogeneous populations of cells. Several studies have demonstrated that these cells can transdifferentiate into cardiomyocytes and vascular-like structures. (4) Unlike embryonic stem cells, MSCs do not spontaneously form cardiomyocytes in vitro, but require stimulation of some form, in order to proceed along a cardiomyocytic lineage.

MSCs have been shown to differentiate into cardiomyocytes and endothelial cells in vivo when transplanted in to the heart, in both non-injury and myocardial infarction models. The cells have been strictly characterized by immunohistochemistry, and positively stain for cardiac and endothelial specific markers, as well as gap junction proteins. (5) Myocardial function and capillary formation are significantly increased in experimental groups treated with MSCs, when compared with controls. (6) The ability of MSCs to transdifferentiate into specialized cells that improve function of the failing heart makes MSCs a realistic option for cellular transplantation.

Endothelial progenitor cells (EPCs) can contribute to tissue revascularization and can be isolated from adult bone marrow or from the peripheral circulation (termed circulating endothelial progenitor cells—CEPs). EPCs can proliferate in vitro to form mature endothelial cells. Human CEPs have also shown potential to differentiate into cardiomyocytes. When co-cultured with neonatal rat cardiomyocytes, human CEPs formed cells with a cardiomyocytic phenotype, as defined by positive staining for cardiac specific markers such as troponin, atrial natriuretic peptide and MEF-2. Functional gap junctions were also demonstrated with transfer of Lucifer yellow dye and calcein between the cells. (7)

Considerable work has been done in recent years to investigate the hypothesis that bone marrow-derived EPCs can home to areas of tissue ischemia and participate in vasculogenesis, thereby increasing blood flow to such areas and potentially preserving or restoring end organ function. In a murine model of myocardial infarction in which bone marrow-derived cells were identifiable by genetic marking, bone marrow-derived EPCs were observed to be incorporated within the endothelium of small vessels in the infarct border zone. (8) In an athymic rat model, human CD34+ cells from donors pretreated with G-CSF were injected into animals that underwent myocardial infarction. (9) Animals that received CD34+ cells had enhanced angiogenesis in the peri-infarct zone and in the infarct border zone, compared with controls. Human endothelial cells were identified within vessels in the center of the infarcted region, suggesting that a population of the injected cells can participate in vasculogenesis after myocardial infarction. Apoptosis in myocytes in the peri-infarct zone was markedly lower in animals receiving CD34+ cells than in control animals.

Individuals with severe peripheral vascular disease and lower-extremity claudication represent an emerging public health concern, and evidence suggests that EPCs may be useful in this form of ischemic insult. Fluorescence-labeled human CD34+ cells injected intravenously into a mouse with hindlimb ischemia were integrated into capillaries and supplied preserved skeletal myocytes six weeks after cell injection. (10) Similarly, ex vivo expanded human EPCs transplanted into nude mice with hindlimb ischemia improved blood flow and capillary density. These mice had a lower rate of limb loss than mice treated with human microvascular endothelial cells. (11) Hindlimb ischemia in the mouse is also a sufficient stimulus to increase the number of endogenous, circulating Sca-1+ (stem cell antigen-1 positive) EPCs. (12)

EPCs may also contribute to cerebral neovascularization after cerebral ischemia. Endothelial cells in the brain are linked by complex junctions that form the blood-brain barrier, and the turnover of endothelial cells in the brain is extremely limited. However, bone marrow-derived EPCs have been detected incorporating into sites of neovascularization around sites of cerebral infarcts in mice, as well as in vessels within the choroid plexus. (13)

In order for bone marrow-derived EPCs to participate in postnatal vasculogenesis or endothelial repair, they respond to signals indicating that they should mobilize from the bone marrow, home to the site of ongoing vascular development, and differentiate into mature endothelial cells.

Vascular endothelial growth factor (VEGF) appears to be an important mediator of EPC mobilization to the peripheral circulation. VEGF expression is markedly increased in hypoxic tissues and tumors, largely because of the effects of hypoxia-inducible factor-1 (HIF-1) on VEGF transcription. Gene knockout experiments have demonstrated the essential role of VEGF in embryonic vasculogenesis, and it is thought to promote sprouting and non-sprouting angiogenesis in adult vascular development.

In animal models, exogenous administration of VEGF promotes rapid mobilization of EPCs into the peripheral circulation.

EPC levels in the bloodstream rise within 24 h after VEGF administration. In patients with severe angina and no options for percutaneous or surgical revascularization, intramyocardial administration of a plasmid encoding VEGF increases the number of EPCs in the peripheral circulation. (14) In patients experiencing vascular trauma in the form of severe burn injury or coronary artery bypass grafting, EPC numbers rise ˜50-fold at 12 h post-injury, and return to baseline by 48-72 h. The kinetics of EPC levels seen in these patients closely mirrors the levels of VEGF detected in the peripheral circulation. (15) In addition, in patients with acute myocardial infarction (MI), levels of circulating CD34+ cells increase one week after MI, and this rise again mirrors the peak in serum VEGF levels. (16) Cytokines that promote granulocyte proliferation and peripheral mobilization of granulocytes may similarly affect EPC mobilization. Increased numbers of EPCs are seen in mice and rabbits treated with granulocyte macrophage colony stimulating factor (GM-CSF), and enhanced neovascularization with bone marrow-derived cells is seen in mice treated with GM-CSF in a corneal neovascularization model. (17)

Another regulator of progenitor cell trafficking is the chemokine stromal cell-derived factor-1 (SDF-1 or CXCL12), which mediates homing of implanted HSCs from peripheral blood to bone marrow, by binding to CXCR4 on circulating cells. SDF-1 and CXCR4 are expressed in complementary patterns during embryonic organogenesis and guide primordial stem cells to sites of rapid vascular expansion. It was also shown that SDF-1 gene expression is regulated by the transcription factor HIF-1 in endothelial cells, resulting in selective in vivo expression of SDF-1 in ischemic tissue in direct proportion to reduced oxygen tension. HIF-1-induced SDF-1 expression increases the adhesion, migration and homing of circulating CXCR4-positive progenitor cells to ischemic tissue. Blockade of SDF-1 in ischemic tissue or CXCR4 on circulating cells prevents progenitor cell recruitment to sites of injury. (18)

Another component of mobilization is the finding that stem and progenitor cell bone marrow niches are locally hypoxic. This idea has been suggested in a previous report indicating that bone marrow aspirates are hypoxic. (19) Direct examination of the bone marrow and uninjured tissues of mice showed that the oxygen tension in the bone marrow compartment in situ was consistently lower than in other tissues and, in fact, very similar to ischemic tissue in an ischemic model. Microscopic analysis showed that the bone marrow compartment contained discrete regions of hypoxia defined by pimonidazole localization that were associated with abundant SDF-1 immunostaining. Systemically-administered EPCs specifically homed to and engrafted these regions regardless of the presence of a peripheral ischemic stimulus. (18) These heterogeneous regions of hypoxia in the bone marrow microenvironment may explain the constitutive and regional expression of SDF-1 in the bone marrow and the CXCR4-dependent stem and progenitor cell tropism to the bone marrow.

The following references are incorporated herein by reference:

1. Orlic D, Kajstura J, Chimenti S, Limana F, Jakoniuk I, Quaini F, Nadal-Ginard B, Bodine D M, Leri A, Anversa P (2001b) Mobilized bone marrow cells repair the infarcted heart, improving function and survival. Proc. Natl. Acad. Sci. USA 98, 10344.

2. Norol F, Merlet P, Isnard R, Sebillon P, Bonnet N, Cailliot C, Carrion C, Ribeiro M, Charlotte F, Pradeau P, Mayol J F, Peinnequin A, Drouet M, Safsafi K, Vernant J P, Herodin F (2003) Influence of mobilized stem cells on myocardial infarct repair in a nonhuman primate model. Blood 102, 4361.

3. Seiler C, Pohl T, Wustmann K, Hutter D, Nicolet P A, Windecker S, Eberli F R, Meier B (2001.) Promotion of collateral growth by granulocyte-macrophage colony-stimulating factor in patients with coronary artery disease: a randomized, double-blind, placebo-controlled study. Circulation 104, 2012.

4. Toma C, Pittenger M F, Cahill K S, Byrne B J, Kessler P D (2002) Human mesenchymal stem cells differentiate to a cardiomyocyte phenotype in the adult murine heart. Circulation 105, 93.

5. Gojo S, Gojo N, Takeda Y, Mori T, Abe H, Kyo S, Hata J, Umezawa A (2003) In vivo cardiovasculogenesis by direct injection of isolated adult mesenchymal stem cells. Exp. Cell Res. 288, 51.

6. Davani S, Marandin A, Mersin N, Royer B, Kantelip B, Herve P, Etievent J P, Kantelip J P (2003) Mesenchymal progenitor cells differentiate into an endothelial phenotype, enhance vascular density, and improve heart function in a rat cellular cardiomyoplasty model. Circulation 108, II253.

7. Badorff C, Brandes R P, Popp R, Rupp S, Urbich C, Aicher A, Fleming I, Busse R, Zeiher A M, Dimmeler S (2003) Transdifferentiation of blood-derived human adult endothelial progenitor cells into functionally active cardiomyocytes. Circulation 107, 1024.

8. Asahara T, Masuda H, Takahashi T, et al. 1999. Bone marrow origin of endothelial progenitor cells responsible for postnatal vasculogenesis in physiological and pathological neovascularization. Circ. Res. 85:221-28.

9. Kocher A A, Schuster M D, Szabolcs M J, et al. 2001. Neovascularization of ischemic myocardium by human bone marrow-derived angioblasts prevents cardiomyocyte apoptosis, reduces remodeling and improves cardiac function. Nat. Med. 7:430-36.

10. Asahara T, Murohara T, Sullivan A, et al. 1997. Isolation of putative progenitor endothelial cells for angiogenesis. Science 275:964-67.

11. Kalka C, Tehrani H, Laudenberg B, et al. 2000. VEGF gene transfer mobilizes endothelial progenitor cells in patients with inoperable coronary disease. Ann. Thorac. Surg. 70:829-34.

12. Asahara T, Takahashi T, Masuda H, et al. 1999. VEGF contributes to postnatal neovascularization by mobilizing bone marrow-derived endothelial progenitor cells. EMBO J. 18:3964-72 Circulation 107:3059-65.

13. Zhang Z G, Zhang L, Jiang Q, et al. 2002. Bone marrow-derived endothelial progenitor cells participate in cerebral neovascularization after focal cerebral ischemia in the adult mouse. Circ. Res. 90:284-88.

14. Kalka C, Tehrani H, Laudenberg B, et al. 2000. VEGF gene transfer mobilizes endothelial progenitor cells in patients with inoperable coronary disease. Ann. Thorac. Surg. 70:829-34 9:1370-76

15. Gill M, Dias S, Hattori K, et al. 2001. Vascular trauma induces rapid but transient mobilization of VEGFR2+AC133+ endothelial precursor cells. Circ. Res. 88:167-74.

16. Shintani S, Murohara T, Ikeda H, et al. 2001. Mobilization of endothelial progenitor cells in patients with acute myocardial infarction. Circulation 103:2776-79.

17. Takahashi T, Kalka C, Masuda H, et al. 1999. Ischemia- and cytokine-induced mobilization of bone marrow-derived endothelial progenitor cells for neovascularization. Nat. Med. 5:434-38.

18. Ceradini D, Kulkarni A, Callaghan M, et al. 2004. Progenitor cell trafficking is regulated by hypoxic gradients through HIF-1 induction of SDF-1. Nature Med 10:858-864.

19. Harrison, J. S., Rameshwar, P., Chang, V. & Bandari, P. 2002. Oxygen saturation in the bone marrow of healthy volunteers. Blood 99, 394.

U.S. Pat. No. 6,810,286 to Donovan et al., which is incorporated herein by reference, describes a subthreshold pulse generator for the local production of angiogenic growth factors, such as vascular endothelial growth factor. The pulse generator is typically configured to be implantable in a patient, in order to reduce or repair tissue injury or disease by regulating angiogenic growth factor production. Alternatively, the subthreshold stimulation provided is sufficient to stimulate angiogenesis in the targeted body tissue. Additionally, a method for pacing is described for stimulating cells or tissues for the controlled expression of angiogenic factors.

U.S. Pat. Nos. 6,569,428 and 5,980,887 to Isner et al., which are incorporated herein by reference, describe pharmaceutical products comprising Endothelial Cell (EC) progenitors for use in methods for regulating angiogenesis, i.e., for enhancing or inhibiting blood vessel formation in a patient and, for some applications, for targeting an angiogenesis modulator to specific locations. For example, the EC progenitors can be used to enhance angiogenesis or to deliver an angiogenesis modulator, e.g., anti- or pro-angiogenic agents, respectively, to sites of pathologic or utilitarian angiogenesis. Additionally, EC progenitors can be used to induce reendothelialization of an injured blood vessel, and thus reduce restenosis by indirectly inhibiting smooth muscle cell proliferation.

U.S. Pat. No. 6,676,937 to Isner et al., which is incorporated herein by reference, describes methods for modulating formation of new blood vessels. In one embodiment, the methods include administering to a mammal an effective amount of granulocyte macrophage-colony stimulating factor (GM-CSF) sufficient to form the new blood vessels. Additionally described are methods for preventing or reducing the severity of blood vessel damage in a mammal, preferably including administering to the mammal an effective amount of GM-CSF.

U.S. Pat. No. 6,767,737 to Wilson et al., which is incorporated herein by reference, describes a composition of substantially purified pluripotent stem cells that are positive both for fibroblast growth factor receptor (FGFR) and a phenotype indicative of a primitive state, such as CD34⁺, CD34⁻lin⁻, Thy-1⁺, AC133⁺ or c-kit⁺. The state of being an embryonic stem cell is also described as being a phenotype indicative of a primitive state.

PCT Publication WO 03/090512 to Itescu, which is incorporated herein by reference, describes a method for treating a disorder of a subject's heart involving loss of cardiomyocytes. The method comprises administering to the subject an amount of an agent effective to cause cardiomyocyte proliferation within the subject's heart so as to thereby treat the disorder. For some applications, the agent is human endothelial progenitor cells, G-CSF, GM-CSF, SDF-1, and IL-8.

The following articles and letters, which are incorporated herein by reference, may be of interest:

Ceradini D J et al., “Progenitor cell trafficking is regulated by hypoxic gradients through HIF-1 induction of SDF-1,” Nature Medicine 10:858-864 (2004)

Penn M S et al., “Role of stem cell homing in myocardial regeneration,” International Journal of Cardiology 95 Suppl. 1:S23-S25 (2004)

Franz W M et al, “Stem-cell homing and tissue regeneration in ischaemic cardiomyopathy,” The Lancet 362:675-676 (2003)

Payne A G, “Using immunomagnetic technology and other means to facilitate stem cell homing,” Medical Hypotheses 62:718-720 (2004).

### SUMMARY OF THE INVENTION

In accordance with an embodiment of the present invention, there is provided a method for facilitating treatment of a patient, including stressing a portion of the patient to an extent sufficient to induce homing of progenitor cells to the portion of the patient.

In an embodiment:

the method includes determining that the portion is in an ischemic state,

wherein stressing the portion includes stressing the portion, following the determination, to an extent sufficient to augment the ischemic state.

In an embodiment, stressing the portion includes transiently augmenting a pathology of the portion of the patient.

In an embodiment, the progenitor cells include endothelial progenitor cells (EPCs).

In an embodiment, stressing includes chemically stressing.

In an embodiment, stressing includes electrically stressing.

In an embodiment, stressing includes mechanically stressing.

In an embodiment, stressing includes inducing inflammation of the portion.

In an embodiment, the method does not include administering the progenitor cells to the patient.

In an embodiment, the method includes facilitating mobilization of the progenitor cells into peripheral blood of the patient.

In an embodiment, facilitating the mobilization includes administering a drug that mobilizes the progenitor cells.

In an embodiment, facilitating the mobilization includes increasing peripheral blood oxygen partial pressure.

In an embodiment, facilitating the mobilization includes inducing transient ischemia at a site of the patient.

In an embodiment, inducing the transient ischemia at the site includes selecting the site to include the portion of the patient.

In an embodiment, inducing the transient ischemia at the site includes selecting the site to not include the portion of the patient.

In an embodiment, inducing the transient ischemia at the site includes selecting the site to include a limb of the patient.

In an embodiment, the method includes administering the progenitor cells to the patient.

In an embodiment, the administered progenitor cells include progenitor cells that have been enriched ex vivo from an original set of progenitor cells extracted from the patient, and the method includes, prior to administering the ex-vivo-enriched progenitor cells to the patient, facilitating mobilization of the original set of progenitor cells into peripheral blood of the patient.

In an embodiment, facilitating the mobilization includes administering a drug that mobilizes the progenitor cells.

In an embodiment, facilitating the mobilization includes increasing peripheral blood oxygen partial pressure.

In an embodiment, stressing the portion includes stressing a heart of the patient. In an embodiment, stressing the heart includes administering a drug that affects the heart. In an embodiment, administering the drug includes administering a tachycardia-inducing drug.

In an embodiment, stressing the portion includes stressing a brain of the patient. In an embodiment, stressing the brain includes reducing blood flow to the brain.

In an embodiment, stressing the portion includes stressing bowel of the patient. In an embodiment, stressing the bowel includes administering a drug that restricts blood flow to the bowel. In an embodiment, stressing the bowel includes increasing peristalsis of the bowel to an extent sufficient to elevate a level of ischemia of the bowel.

There is further provided, in accordance with an embodiment of the present invention, a method for use with tissue of a patient, including:

evaluating an indication of a level of stromal cell-derived factor-1 (SDF-1) in the tissue; and

determining an indication of a number of stem cells in the tissue responsive to the indication of the level of SDF-1.

In an embodiment, tissue includes blood extracted from the patient, and wherein determining includes determining an indication of a number of stem cells in the extracted blood.

In an embodiment, the method includes diagnosing a condition of the patient responsive to the indication of the level of SDF-1.

In an embodiment, the method includes increasing ex vivo a number of progenitor cells obtained from a patient blood sample that was extracted following the determining of the indication of the number of stem cells.

There is yet further provided, in accordance with an embodiment of the present invention, a method for use with tissue of a patient, including:

evaluating an indication of a level of stromal cell-derived factor-1 (SDF-1) in the tissue; and

determining an indication of a level of stress of a portion of the patient, responsive to the indication of the level of SDF-1.

In an embodiment, the method includes determining a time for administration of progenitor cells to the patient, responsive to the level of stress of the portion of the patient.

In an embodiment, the portion is selected from the list consisting of: heart, bowel, limb, and brain, and wherein determining the indication includes determining an indication of a level of stress of the selected portion.

In an embodiment, the method includes determining a time for administration of progenitor cells to the patient, to a site local to the portion, responsive to the indication of the level of stress of the portion of the patient.

In an embodiment, the site local to the portion is a transcatheter administration site of the progenitor cells.

In an embodiment, the method includes determining a time for administration of progenitor cells to the patient, at a site remote from the portion, responsive to the indication of the level of stress of the portion of the patient.

In an embodiment, the site remote from the portion is an intravenous administration site of the progenitor cells.

### DETAILED DESCRIPTION OF EMBODIMENTS

In accordance with some embodiments of the present invention, a method is provided to influence the natural healing process of damaged tissue. For example, changes in the physiological environment inside and around damaged tissue can be configured to accelerate or attenuate the healing process.

In accordance with an embodiment of the present invention, a method is provided to create reversible physiological changes in a specific body region, such as bone marrow, heart, brain, kidney, eye, endocrine glands, bowel, or limbs. These physiological changes modulate (e.g., augment) the production and secretion of several control factors. These factors in turn influence the healing processes.

In accordance with an embodiment of the present invention, a stressful condition is induced, typically in a specific organ. The stressful condition may be induced in one or more of several ways, such as by manipulating the blood supply to the organ, increasing its oxygen demands, and/or inducing an inflammatory reaction (such as a well-controlled, low grade inflammatory reaction).

In accordance with an embodiment of the present invention, methods for stressing an organ are practiced in combination with a stem cell implantation procedure, typically in order to further enhance treatment of a disease.

For some applications, methods described herein are configured to improve the state of the heart of a patient. For example, a physician may actively enrich peripheral blood oxygen partial pressure (PO2), even beyond the stage when hemoglobin molecules are determined to be essentially completely saturated with oxygen. Typically, the excess oxygen is dissolved in the blood. Thus, blood PO2 is higher than in a healthy patient at rest. The oxygen enrichment is typically performed for about 4 to about 36 hours (e.g., about 8 to about 16 hours, for example about 12 hours). The oxygen enrichment typically leads to lowering of SDF-1 production in bone marrow and enhances mobilization of progenitor cells into the peripheral blood.

In combination with the elevation of blood PO2, or separately therefrom, IV administration of cardiac chronotropic drug (e.g., atropine) is initiated, which in turn creates a tachycardia that induces local ischemia in the heart. The cardiac ischemia leads to local production of HIF-1 and SDF-1, and homing of the progenitor cells to the heart.

Independently or in combination with therapies described herein, high levels of HIF-1 and/or SDF-1 are used in a diagnostic procedure as an indicator of the extent of tissue ischemia.

Typically, the ischemically-derived enhanced local production of HIF-1 and SDF-1, as provided by these embodiments, enhances mobilization of progenitor cells. For some applications, peripheral blood is collected, and ex vivo separation and enrichment of progenitor cells is performed. In this case, the optional administration of tachycardia- and ischemia-inducing IV atropine is typically performed following attaining a large number of ex vivo progenitor cells, e.g., about 4 million to about 50 million cells. During or following the tachycardia/ischemia event, the enriched progenitor cell population is administered to the patient, typically either intravenously or via a catheter placed in or adjacent to a desired target site, such as a coronary artery. For example, the progenitor cells may be administered to the patient about 4 to about 36 hours (e.g., 12 hours) following the administration of the atropine.

For some applications, dipyridamole or another coronary vasodilator is administered, instead of or in addition to the atropine. Under the influence of dipyridamole, ischemic blood vessels receive relatively less blood flow than non-ischemic blood vessels, thereby transiently enhancing the ischemia and increasing homing of progenitor cells to the ischemic tissue.

In combination with the techniques described hereinabove, or separately therefrom, SDF-1 may be injected directly into the patient, either intravenously or at a particular ischemic target site.

For some applications, methods described herein are configured to treat peripheral vascular disease of a patient. Typically, enrichment of peripheral blood oxygen saturation is performed, as described hereinabove, leading to lowering of SDF-1 production in bone marrow and enhancing mobilization from the bone marrow of progenitor cells. Creation of controlled local ischemia in the limbs is performed, for example, by intermittent arterial occlusion for 15-30 minute intervals every hour, during each of four consecutive hours. It is to be appreciated that other techniques for inducing limb ischemia are considered to be within the scope of the present invention. The induced ischemia leads to local production of HIF-1 and SDF-1, which further enhances mobilization of progenitor cells.

For some applications, peripheral blood is collected, and ex vivo separation of the blood is performed, followed by enrichment of progenitor cells. Typically, the enriched suspension of progenitor cells is injected into the gastrocnemius muscles or into another site in the vicinity of the ischemic tissue.

For some applications, following ex vivo expansion of the progenitor cells, controlled local ischemia of the limbs is induced, e.g., by intermittent arterial occlusion for 15-30 minute intervals every hour, for six consecutive hours. The ischemia leads to local production of HIF-1 and SDF-1, which enhances homing of progenitor cells. Injection of the enriched suspension of the progenitor cells into distal peripheral arteries may be facilitated, as appropriate, by angiography. For example, the enriched suspension of progenitor cells may be injected into the popliteal artery, or into a site further distal from the heart.

As noted above, high levels of HIF-1 and/or SDF-1 in peripheral blood may be used as an indicator of the level of tissue ischemia.

For some applications, methods described herein are configured to treat a bowel condition of a patient (e.g., mesenteric angina). Enrichment of oxygen saturation in peripheral blood is typically performed, as described hereinabove, e.g., for 12 hours, leading to lowering of SDF-1 production in bone marrow, and enhancing mobilization of progenitor cells. Alternatively or additionally, controlled local ischemia in one or both limbs is induced by intermittent arterial occlusion (e.g., for 15-30 minute intervals every hour, for four consecutive hours). The limb ischemia leads to local production of HIF-1 and SDF-1, which further enhances mobilization of progenitor cells.

As appropriate, collection of peripheral blood and ex vivo separation and enrichment of progenitor cells may be performed, as described hereinabove. Subsequently, creation of controlled local ischemia in the bowel is typically performed by administration of Terlipressin, Octreotide or Vasopressin (which are all splanchnic vessel contractors). Alternatively or additionally, ischemia is induced by administering a cholinergic and/or an anticholinesterase agent (e.g., physostigmine), at a dosage which induces a high level of bowel peristalsis. Typically, within one day of administration of one or more of these drugs, the enriched suspension of progenitor cells is injected into the superior mesenteric artery, usually facilitated by angiography.

As appropriate, high levels of HIF-1 and SDF-1 in peripheral blood may be used as indicators of the level of ischemia.

For some applications, methods described herein are configured to treat a condition of a brain of a patient. As described hereinabove, enrichment of oxygen saturation in peripheral blood is typically performed, leading to lowering of SDF-1 production in bone marrow and enhancing mobilization of progenitor cells. Alternatively or additionally, controlled local ischemia in one or both limbs is induced by intermittent arterial occlusion (e.g., for 15 minute intervals every hour, for four consecutive hours). The ischemia leads to local production of HIF-1 and SDF-1, which further enhances mobilization of progenitor cells.

As appropriate, collection of peripheral blood and ex vivo separation and enrichment of progenitor cells may be performed. Creation of controlled local ischemia in the brain may be performed by lowering systolic blood pressure to 80 mm/hg. Administration of the enriched suspension of the progenitor cells by angiography into carotid artery.

For some applications, high levels of HIF-1 and SDF-1 in peripheral blood are used as indicators of the level of ischemia.

Typically, but not necessarily, the progenitor cells described herein include EPCs. Alternatively, they include progenitor cells which are not EPCs.

In an embodiment, techniques described herein are practiced in combination with (a) techniques described in one or more of the references cited herein, (b) techniques described in U.S. Provisional Patent Application 60/576,266, filed Jun. 1, 2004, and/or (c) techniques described in U.S. Provisional Patent Application 60/588,520, filed Jul. 15, 2004. Both of these provisional patent applications are assigned to the assignee of the present patent application and are incorporated herein by reference.

For example, techniques described in the present patent application may be practiced in combination with the following methods for isolation, differentiation and expansion of stem cells from a tissue. For example, the stem cells may include endothelial progenitor cells (EPCs). Alternatively or additionally, the tissue may include human peripheral blood. Typically, the stem cells are transplanted into the donor or into another individual (e.g., in order to enhance vasculogenesis and/or angiogenesis and/or neovascularization). The present patent application provides protocols for obtaining a product containing appropriate numbers of functional EPCs. The methods described include: (a) EPC isolation; (b) culture of cells for 3-30 days in enriched culture medium; and/or (c) implantation of appropriate number of EPCs into a patient. It is to be understood that whereas some embodiments described herein relate specifically to EPCs derived from blood, the scope of the present invention includes techniques for use with stem cells derived from a variety of body tissues, mutatis mutandis.

For some applications, the method comprises collecting a blood sample from a donor and/or a patient, isolating from the sample peripheral blood mononuclear cells, separating a population of cells rich in monocytes and progenitor cells from the mononuclear cell fraction, and growing these cells under conditions that will cause the hematopoietic progenitor cells present in the mixture of cells to differentiate into EPCs and proliferate. This ex vivo expansion step is typically utilized because the number of EPCs in the circulation is below 0.1%. Following this augmentation stage, the cells may be implanted by injection into the coronary vessels or into the myocardium of a patient.

There is therefore provided, in accordance with an embodiment of the present invention, a method for use with extracted blood, including:

applying blood to a first gradient suitable for selecting first-pass cells having a density less than 1.077 g/ml;

applying the first-pass cells to a second gradient suitable for selecting second-pass cells having a density between 1.030 and 1.068 g/ml; and

increasing the number of cells having a density between 1.030 and 1.068 g/ml, by culturing the second-pass cells for a period lasting between 3 and 30 days.

In an embodiment, applying the blood cells to the first gradient includes applying the blood cells to a Ficoll-like gradient.

In an embodiment, applying the first-pass cells to the second gradient includes applying the first-pass cells to a Percoll-like gradient.

In an embodiment, applying the first-pass cells to the second gradient includes applying the first-pass cells to an OptiPrep-like gradient.

There is further provided, in accordance with an embodiment of the present invention, a method for use with extracted stem cells, including:

applying tissue including the stem cells to a first gradient suitable for selecting first-pass cells having a density less than 1.077 g/ml;

applying the first-pass cells to a second gradient suitable for selecting second-pass cells having a density between 1.030 and 1.068 g/ml;

applying the second-pass cells to a third gradient suitable for selecting third-pass cells having a density between 1.032 and 1.064 g/ml; and

increasing the number of cells having a density between 1.032 and 1.064 g/ml, by culturing the third-pass cells for a period lasting between 3 and 30 days.

In an embodiment, the third gradient is suitable for selecting cells having a density between 1.030 and 1.068 g/ml, and wherein applying the second-pass cells to the third gradient includes selecting the cells having a density between 1.032 and 1.064 g/ml.

There is also provided, in accordance with an embodiment of the present invention, a method for use with extracted blood, including:

applying blood to a first gradient suitable for selecting first-pass cells having a density less than 1.077 g/ml;

applying the first-pass cells to a second gradient suitable for selecting second-pass cells having a density between 1.030 and 1.068 g/ml; and

incubating the second-pass cells on a surface including an antibody.

There is additionally provided, in accordance with an embodiment of the present invention, a method for use with extracted blood, including:

applying blood to a first gradient suitable for selecting first-pass cells having a density less than 1.077 g/ml;

applying the first-pass cells to a second gradient suitable for selecting second-pass cells having a density between 1.030 and 1.068 g/ml; and

incubating the second-pass cells on a surface including a growth-enhancing molecule other than collagen or fibronectin.

In an embodiment, incubating the second-pass cells includes incubating the second-pass cells on a surface that includes, in addition to the growth-enhancing molecule, at least one of: collagen and fibronectin.

There is yet additionally provided, in accordance with an embodiment of the present invention, a method for use with extracted blood, including:

applying blood to a first gradient suitable for selecting first-pass cells having a density less than 1.077 g/ml;

applying the first-pass cells to a second gradient suitable for selecting second-pass cells having a density between 1.030 and 1.068 g/ml; and

culturing the second-pass cells for a period lasting between 1 and 5 days in a culture medium including less than 5% serum.

There is still additionally provided, in accordance with an embodiment of the present invention, a method for use with extracted blood, including:

applying blood to a first gradient suitable for selecting first-pass cells having a density less than 1.077 g/ml;

applying the first-pass cells to a second gradient suitable for selecting second-pass cells having a density between 1.030 and 1.068 g/ml; and

culturing the second-pass cells for a period lasting between 1 and 5 days in a culture medium including greater than or equal to 10% serum.

In an embodiment, culturing the second-pass cells includes culturing the second-pass cells in a culture medium including less than 20% serum.

There is also provided, in accordance with an embodiment of the present invention, a method for use with extracted blood, including:

applying blood to a first gradient suitable for selecting first-pass cells having a density less than 1.077 g/ml;

applying the first-pass cells to a second gradient suitable for selecting second-pass cells having a density between 1.030 and 1.068 g/ml;

during a low-serum time period, culturing the second-pass cells in a culture medium including less than 10% serum; and

during a high-serum time period, culturing the second-pass cells in a culture medium including greater than 10% serum.

In an embodiment, culturing the second-pass cells during the low-serum time period includes culturing the second-pass cells for a duration of between 1 and 5 days.

In an embodiment, culturing the second-pass cells during the high-serum time period includes culturing the second-pass cells for a duration of between 1 and 30 days.

In an embodiment, culturing the second-pass cells during the low-serum time period is performed prior to culturing the second-pass cells during the high-serum time period.

In an embodiment, culturing the second-pass cells during the low-serum time period is performed following culturing the second-pass cells during the high-serum time period.

There is further provided, in accordance with an embodiment of the present invention, a method for use with extracted blood, including:

applying blood to a first gradient suitable for selecting first-pass cells having a density less than 1.077 g/ml;

applying the first-pass cells to a second gradient suitable for selecting second-pass cells having a density between 1.030 and 1.068 g/ml;

during a hypoxic time period lasting at least 2 hours, culturing the second-pass cells under hypoxic conditions; and during a non-hypoxic time period lasting at least 1 day, culturing the second-pass cells under non-hypoxic conditions.

In an embodiment, the hypoxic and non-hypoxic time-periods are within a culturing time period lasting less than 30 days, and culturing the second-pass cells under hypoxic conditions includes culturing the second-pass cells under hypoxic conditions during a first two days of the culturing time period.

In an embodiment, the hypoxic and non-hypoxic time-periods are within a culturing time period lasting less than 30 days, and culturing the second-pass cells under hypoxic conditions includes culturing the second-pass cells under hypoxic conditions during a last two days of the culturing time period.

In an embodiment, the hypoxic and non-hypoxic time-periods are within a culturing time period lasting less than 30 days, and culturing the second-pass cells under hypoxic conditions includes culturing the second-pass cells under hypoxic conditions for at least 2 hours between a first two days and a last two days of the culturing time period.

In an embodiment, culturing the second-pass cells under hypoxic conditions is performed prior to culturing the second-pass cells under non-hypoxic conditions.

In an embodiment, culturing the second-pass cells under hypoxic conditions is performed following culturing the second-pass cells under non-hypoxic conditions.

There is still further provided, in accordance with an embodiment of the present invention, a method for use with extracted blood, including:

applying blood to a first gradient suitable for selecting first-pass cells having a density less than 1.077 g/ml;

applying the first-pass cells to a second gradient suitable for selecting second-pass cells having a density between 1.030 and 1.068 g/ml; and

culturing the second-pass cells in a culture medium including at least one of the following: erythropoietin, statin molecules, and an antidiabetic agent.

In an embodiment, the antidiabetic agent includes Rosiglitazone, and culturing the second-pass cells includes culturing the second-pass cells in a culture medium including Rosiglitazone.

There is yet further provided, in accordance with an embodiment of the present invention, a method for use with extracted stem cells, including:

applying tissue including the stem cells to a first gradient suitable for selecting first-pass cells having a density less than 1.077 g/ml;

applying the first-pass cells to a second gradient suitable for selecting second-pass cells having a density between 1.030 and 1.068 g/ml; and

increasing the number of cells having a density between 1.030 and 1.068 g/ml, by culturing the second-pass cells for a period lasting between 3 and 30 days.

In an embodiment, the method includes extracting the stem cells from bone marrow.

In an embodiment, the method includes mobilizing the stem cells from bone marrow to facilitate extraction of the stem cells.

In an embodiment, the method includes extracting the stem cells from blood.

In an embodiment, culturing the second-pass cells includes:

culturing the second-pass cells in a first container during a first portion of the period;

removing at least some of the second-pass cells from the first container at the end of the first portion of the period; and

culturing, in a second container during a second portion of the period, the cells removed from the first container.

In an embodiment, removing the at least some of the second-pass cells includes selecting for removal cells that adhere to a surface of the first container.

In an embodiment, removing the at least some of the second-pass cells includes selecting for removal cells that do not adhere to a surface of the first container.

In an embodiment, the first container includes on a surface thereof a growth-enhancing molecule, and culturing the cells in the first container includes culturing the cells in the first container that includes the growth-enhancing molecule.

In an embodiment, the second container includes on a surface thereof a growth-enhancing molecule, and culturing the cells in the second container includes culturing the cells in the second container that includes the growth-enhancing molecule.

In an embodiment, the growth-enhancing molecule is selected from the list consisting of: collagen, fibronectin, a growth factor, and an antibody to a stem cell surface receptor.

In accordance with an embodiment of the present invention, a method is provided for isolating, differentiating, and growing endothelial progenitor cells (EPCs) from human peripheral blood. The EPCs are typically implanted in a patient to induce vasculogenesis and/or angiogenesis and/or neovascularization. Typically, peripheral blood mononuclear cells (PBMCs) separated by Ficoll are further enriched by one or more other density gradients (such as Percoll, OptiPrep, or Nycodenz), and are then allowed to adhere to tissue culture dishes. Cells are typically grown for 3-30 days in an enriched culture medium. At several time points during the culture period, samples are taken for phenotypic assessment. Expanded cells are collected and saved until implantation into the patient.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for facilitating treatment of a patient, comprising stressing a portion of the patient to an extent sufficient to induce homing of progenitor cells to the portion of the patient.

2. The method according to claim 1,
comprising determining that the portion is in an ischemic state,
wherein stressing the portion comprises stressing the portion, following the determination, to an extent sufficient to augment the ischemic state.

3. The method according to claim 1, wherein stressing the portion comprises transiently augmenting a pathology of the portion of the patient.

4. The method according to claim 1, wherein the progenitor cells include endothelial progenitor cells (EPCs).

5. The method according to claim 1, wherein stressing comprises chemically stressing.

6. The method according to claim 1, wherein stressing comprises electrically stressing.

7. The method according to claim 1, wherein stressing comprises mechanically stressing.

8. The method according to claim 1, wherein stressing comprises inducing inflammation of the portion.

9. The method according to claim 1, not comprising administering the progenitor cells to the patient.

10. The method according to claim 9, comprising facilitating mobilization of the progenitor cells into peripheral blood of the patient.

11. The method according to claim 10, wherein facilitating the mobilization comprises administering a drug that mobilizes the progenitor cells.

12. The method according to claim 10, wherein facilitating the mobilization comprises increasing peripheral blood oxygen partial pressure.

13. The method according to claim 10, wherein facilitating the mobilization comprises inducing transient ischemia at a site of the patient.

14. The method according to claim 13, wherein inducing the transient ischemia at the site comprises selecting the site to include the portion of the patient.

15. The method according to claim 13, wherein inducing the transient ischemia at the site comprises selecting the site to not include the portion of the patient.

16. The method according to claim 13, wherein inducing the transient ischemia at the site comprises selecting the site to include a limb of the patient.

17. The method according to claim 1, comprising administering the progenitor cells to the patient.

18. The method according to claim 17,
wherein the administered progenitor cells comprise progenitor cells that have been enriched ex vivo from an original set of progenitor cells extracted from the patient,
and comprising, prior to administering the ex-vivo-enriched progenitor cells to the patient, facilitating mobilization of the original set of progenitor cells into peripheral blood of the patient.

19. The method according to claim 18, wherein facilitating the mobilization comprises administering a drug that mobilizes the progenitor cells.

20. The method according to claim 18, wherein facilitating the mobilization comprises increasing peripheral blood oxygen partial pressure.

21. The method according to claim 1, wherein stressing the portion comprises stressing a heart of the patient.

22. The method according to claim 21, wherein stressing the heart comprises administering a drug that affects the heart.

23. The method according to claim 22, wherein administering the drug comprises administering a tachycardia-inducing drug.

24. The method according to claim 1, wherein stressing the portion comprises stressing a brain of the patient.

25. The method according to claim 24, wherein stressing the brain comprises reducing blood flow to the brain.

26. The method according to claim 1, wherein stressing the portion comprises stressing bowel of the patient.

27. The method according to claim 26, wherein stressing the bowel comprises administering a drug that restricts blood flow to the bowel.

28. The method according to claim 26, wherein stressing the bowel comprises increasing peristalsis of the bowel to an extent sufficient to elevate a level of ischemia of the bowel.

29. A method for use with tissue of a patient, comprising:
evaluating an indication of a level of stromal cell-derived factor-1 (SDF-1) in the tissue; and
determining an indication of a number of stem cells in the tissue responsive to the indication of the level of SDF-1.

30. The method according to claim 29, wherein the tissue includes blood extracted from the patient, and wherein determining comprises determining an indication of a number of stem cells in the extracted blood.

31. The method according to claim 29, comprising diagnosing a condition of the patient responsive to the indication of the level of SDF-1.

32. The method according to claim 29, comprising increasing ex vivo a number of progenitor cells obtained from a patient blood sample that was extracted following the determining of the indication of the number of stem cells.

33. A method for use with tissue of a patient, comprising:
evaluating an indication of a level of stromal cell-derived factor-1 (SDF-1) in the tissue; and
determining an indication of a level of stress of a portion of the patient, responsive to the indication of the level of SDF-1.

34. The method according to claim 33, comprising determining a time for administration of progenitor cells to the patient, responsive to the level of stress of the portion of the patient.

35. The method according to claim 33, wherein the portion is selected from the list consisting of: heart, bowel, limb, and brain, and wherein determining the indication comprises determining an indication of a level of stress of the selected portion.

36. The method according to claim 33, comprising determining a time for administration of progenitor cells to the patient, to a site local to the portion, responsive to the indication of the level of stress of the portion of the patient.

37. The method according to claim 36, wherein the site local to the portion is a transcatheter administration site of the progenitor cells.

38. The method according to claim 33, comprising determining a time for administration of progenitor cells to the patient, at a site remote from the portion, responsive to the indication of the level of stress of the portion of the patient.

39. The method according to claim 38, wherein the site remote from the portion is an intravenous administration site of the progenitor cells.
